(19) United States
(12) Patent Application Publication
(10) Pub. No.: US 2016/0251296 A1
(43) Pub. Date: Sep. 1, 2016

(54) PROCESS TO PREPARE LEVULINIC ACID

(71) Applicant: GFBIOCHEMICALS LIMITED, Valletta (MT)

(72) Inventors: Brian D. Mullen, Delano, MN (US); Cora M. Leibig, Maple Grove, MN (US)

(73) Assignee: GFBiochemicals Limited, Valletta (MT)

(21) Appl. No.: 15/027,960

(22) PCT Filed: Oct. 6, 2014
(86) PCT No.: PCT/US14/59221

§ 371 Date: Apr. 7, 2016

Related U.S. Application Data
(60) Provisional application No. 61/887,657, filed on Oct. 7, 2013.

(51) Int. Cl. C07C 51/44; C07C 51/43

(52) U.S. Cl. CPC C07C 51/44; C07C 51/43

(57) ABSTRACT

The invention describes processes to prepare levulinic acid, formic acid and/or hydroxymethyl furfural from various biomass materials.

### CROSS REFERENCE TO RELATED APPLICATIONS

None

### FIELD OF THE INVENTION

The invention relates generally to the preparation and purification of levulinic acid.

### BACKGROUND OF THE INVENTION

Levulinic acid can be used to make resins, plasticizers, specialty chemicals, herbicides and as a flavor substance. Levulinic acid is useful as a solvent, and as a starting material in the preparation of a variety of industrial and pharmaceutical compounds such as diphenolic acid (useful as a component of protective and decorative finishes), calcium levulinate (a form of calcium for intravenous injection used for calcium replenishment and for treating hypocalcemia. The use of the sodium salt of levulinic acid as a replacement for ethylene glycols as an antifreeze has also been proposed.

Esters of levulinic acid are known to be useful as plasticizers and solvents, and have been suggested as fuel additives. Acid catalyzed dehydration of levulinic acid yields alpha-angelica lactone.

Levulinic acid has been synthesized by a variety of chemical methods. But levulinic acid has not attained much commercial significance due in part to the high cost of the raw materials needed for synthesis. Another reason is the low yields of levulinic acid obtained from most synthetic methods. Yet, another reason is the formation of a formic acid byproduct during synthesis and its separation from the levulinic acid. Therefore, the production of levulinic acid has had high associated equipment costs. Despite the inherent problems in the production of levulinic acid, however, the reactive nature of levulinic acid makes it an ideal intermediate leading to the production of numerous useful derivatives.

Cellulose-based biomass, which is an inexpensive feedstock, can be used as a raw material for making levulinic acid. The supply of sugars from cellulose-containing plant biomass is immense and replenishable. Most plants contain cellulose in their cell walls. For example, cotton comprises 90% cellulose. Furthermore, it has been estimated that roughly 75% of the approximate 24 million tons of biomass generated on cultivated lands and grasslands are waste. The cellulose derived from plant biomass can be a suitable source of sugars to be used in the process of obtaining levulinic acid. Thus, the conversion of such waste material into a useful chemical, such as levulinic acid, is desirable.

### BRIEF SUMMARY OF THE INVENTION

A major issue in producing levulinic acid is the separation of pure levulinic acid from the byproducts, especially from formic acid and char. Current processes generally require high temperature reaction conditions, generally long digestion periods of biomass, specialized equipment to withstand hydrolysis conditions, and as a result, the yield of the levulinic acid is quite low, generally in yields of 10 percent or less.

Therefore, a need exists for a new approach that overcomes one or more of the current disadvantages noted above.

The present invention surprisingly provides novel approaches to more efficiently prepare levulinic acid in commercial quantities with high yields and high purities. Additionally, the production of hydroxymethylfurfural is also described, which is an important intermediate to the product of levulinic acid.

In one aspect, the use of a water insoluble cosolvent in the processes improves the yields of the hydroxymethylfurfural or levulinic acid and helps to reduce undesired byproducts. In another aspect, the use of high concentration of acid, e.g., about 20-50 weight percent based on the total weight of reaction components and low reaction temperature (approximately 50-100° C.) helps to improve the yield of desired products with reduction of undesired byproducts.

In one aspect, hydroxymethyl furfurfal (HMF) can be prepared first followed by a second step to prepare the levulinic acid.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1a is a flow diagram of one embodiment for a process to prepare and/or purify levulinic acid.

FIG. 1b is a flow diagram of another embodiment for a process to prepare and/or purify levulinic acid.

FIGS. 2a through 2e provide information regarding recovery of levulinic acid from Char; soluble and insoluble fractions. It was surprisingly found that extraction of the char provided levulinic acid almost exclusively, helping to further improve the production of levulinic acid.

FIG. 3 provides an aspen flowsheet diagram depicting various reactor configurations.

FIG. 4 depicts an industrial scale process to produce levulinic acid.

FIGS. 5a through 5c are pictures showing reactor components after production of levulinic acid in accordance with the present invention.

FIGS. 5d through 5g are pictures showing reactor components after production of levulinic acid in accordance with the prior art.

### DETAILED DESCRIPTION

In the specification and in the claims, the terms “including” and “comprising” are open-ended terms and should be interpreted to mean “including, but not limited to . . . .” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The present invention provides various advantages in the preparation of levulinic acid (LA), hydroxymethyl furfural (HIVIF) and/or formic acid (FA). The following list of advantages is not meant to be limiting but highlights some of the discoveries contained herein.

First, a biomass material can be used as the initial feedstock to prepare the levulinic acid, hydroxymethyl furfural and/or formic acid. This ability provides great flexibility in obtaining a constant source of starting material and is not limiting.

Second, the biomass can be a refined material, such as fructose, glucose, sucrose, mixtures of those materials and the like. As such, there is a plentiful supply of materials that can be converted into the ultimate product(s). For example, sugar beets or sugar cane can be used as one source. Fructose-corn syrup is another readily available material. Use of such materials thus helps to reduce the costs to prepare the desired products.

Third, it has been discovered that use of high concentrations of acid(s), generally about 20 weight percent or more (based on the total mass of the reaction medium) provides a cleaner reaction product with less char and unwanted byproducts. It has also been found that use of high concentrations of acid(s), generally up to 75 weight percent or more, (based on the total mass of the reaction medium) provides faster reaction times than lower acid concentrations used with the same reaction conditions.

Fourth, it has also been discovered that with the use of higher concentrations of acid, the reaction conditions can be conducted at much lower temperatures than are currently utilized in the literature. Again, this lessens the amount of char and byproducts from the reaction(s) that take place and increases the yield of the desired product(s).

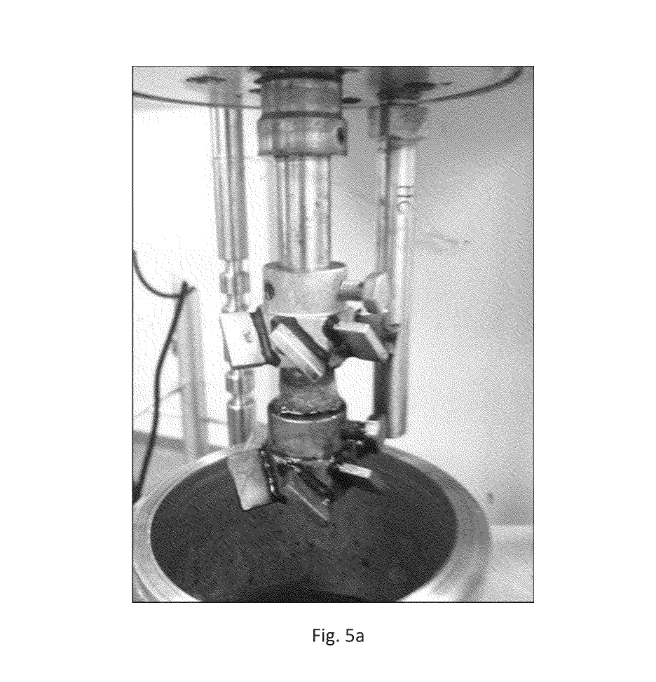

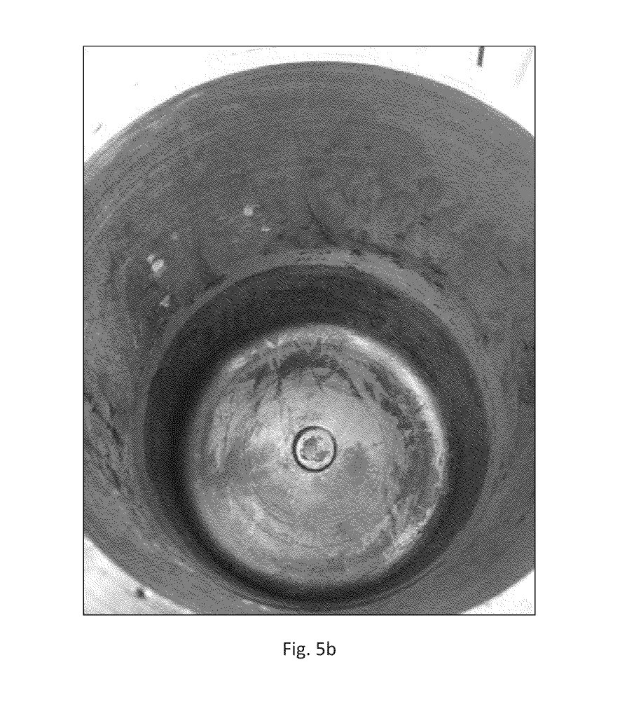

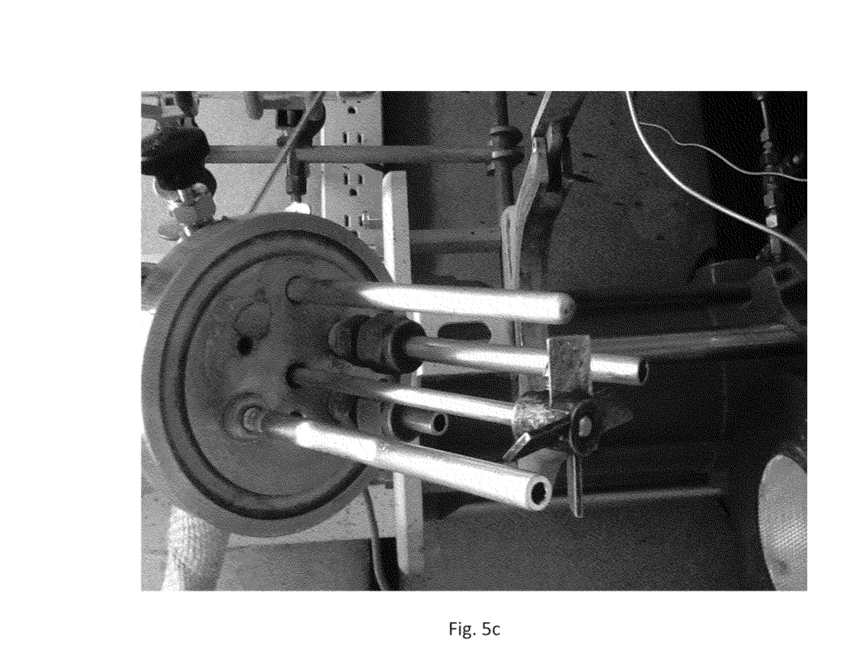

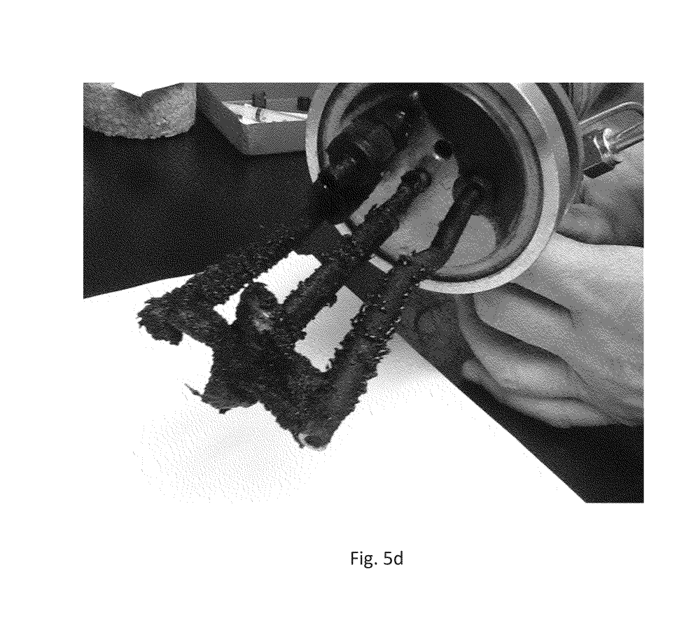

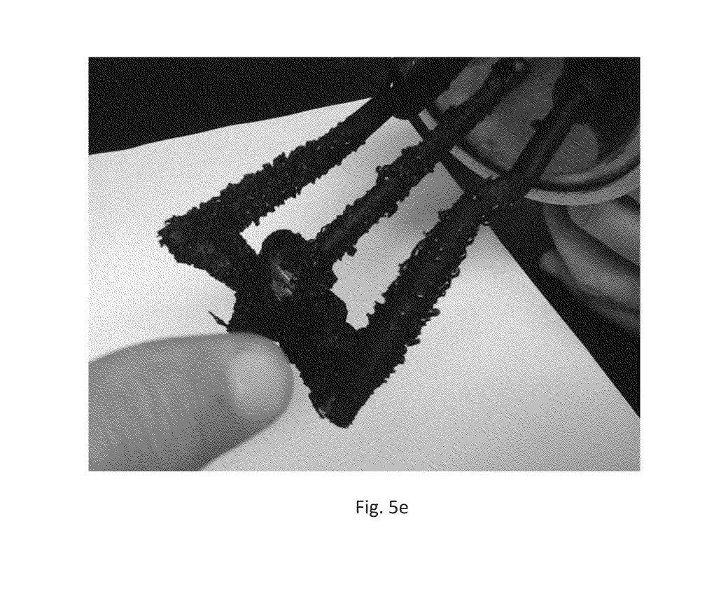

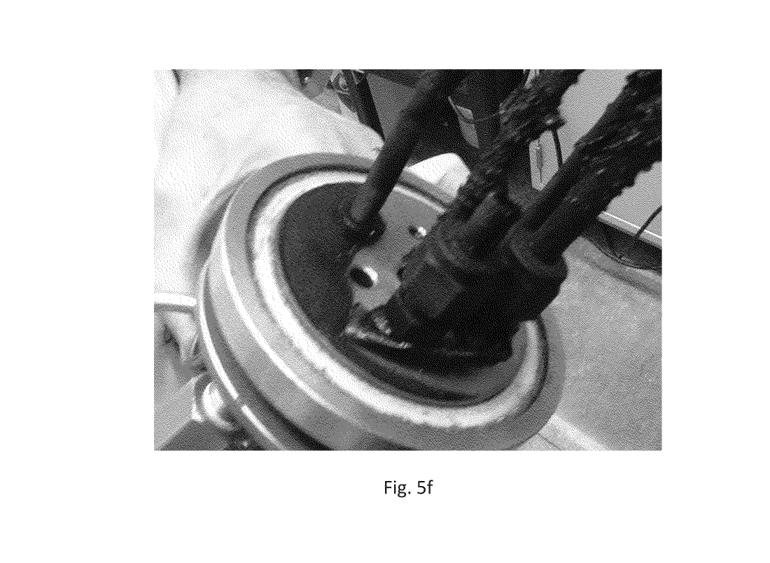

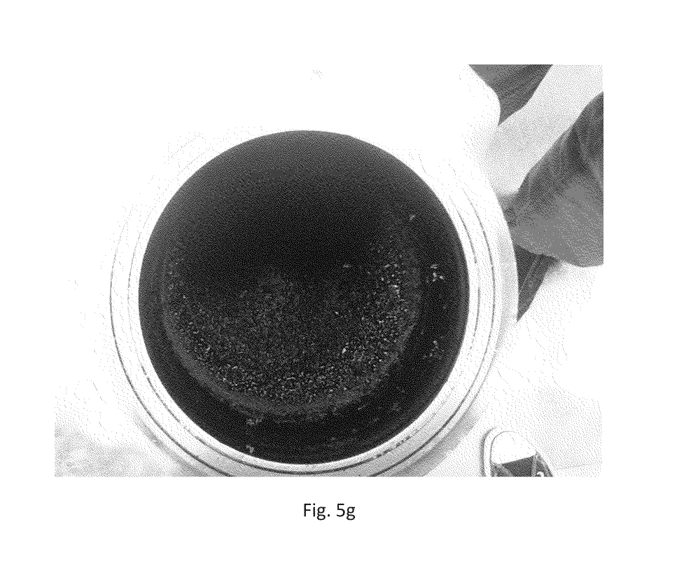

Fifth, it has also been discovered that with the methods of the present invention, the char that is created is much easier to remove from the reactor. For example, FIGS. 5a, 5b and 5c depict internal PARR reactor components after carrying out methods according to the present invention with no additional cleaning. As can be seen in the photographs, there is little to no char accumulated on the reactor components. In comparison, FIGS. 5d through 5g depict internal PARR reactor components after carrying out methods according to the prior art with no additional cleaning. As can be seen, there is significant char build up on the reactor components requiring large cleanup efforts.

Sixth, it has been advantageously found to treat the biomass material(s) in an aqueous environment with a water immiscible solvent. Not to be limited by theory, it is believed that the partitioning of the starting materials from the product(s) between the aqueous and non-aqueous layers provides for one or more of: increased yield, reduced charring and/or by-products, faster reaction times and reduced reaction temperatures.

Seventh, it has also been found that the advantages of the new process conditions, including continuous addition of the biomass over a period of time during the reaction can be incorporated into existing processes to improve yield, reduce costs, improve efficiency and improve purity of product(s).

Eighth, the processes described herein can be performed via CSTR or continuous batch process conditions.

In one embodiment, This process uses a high concentration of sulfuric acid, which has several distinct advantages. For one, the reactions can be run at lower temperatures compared to low acid processes and still hydrolyze the sugars in a reasonable time frame. It has been discovered that under these high acid, low-temperature reaction conditions (e.g., 80 C-110° C.), the char byproduct that is formed is in the form of suspended particles that are easier to remove from the reactor and that can be filtered from the liquid hydrolysate product stream. In contrast, with low acid conditions, high temperature is required to effectively hydrolyze the sugar in a reasonable time frame and those conditions produce a char byproduct that coats the reactor components in such a manner that it is difficult to remove, and for the most part does not stay suspended in the reaction mixture. This high-acid reaction strategy, however, makes it difficult to isolate the organic acid products (levulinic acid and formic acid) from the inorganic acid reagent. When small amounts of sulfuric acid are used, as is typical in the prior art, the strong inorganic acid can effectively be neutralized to its salt form by careful addition of stoichiometric amounts of base. At the high acid contents used here, however, the quantity of salt produced would be excessive. Likewise, the use of an ion exchange column is impractical because the large quantity of inorganic acid would quickly fill the capacity of the column.

Solvent extraction techniques, where the organic acids are preferably extracted into an organic solvent, are preferred. Even here, the high mineral acid content poses challenges. The organic solvent should be insoluble in the aqueous phase, but in some cases, the sulfuric acid can drive compatibility of the organic solvent and the aqueous phase. When this happens, a portion of the organic solvent becomes soluble in the concentrated sulfuric acid aqueous phase and the risk of solvent loss to side reactions increases. Even if the organic solvent is stable in the aqueous sulfuric acid phase, the organic solvent must be recovered from the aqueous stream for recycling to the extraction unit for optimized economics. High mineral acid concentration also carries with it the potential for higher mineral acid concentrations in the organic phase. When this happens, there is the risk of solvent loss to side reactions with the mineral acid, particularly in the case when the organic stream is heated to distill the organic solvent. Therefore, solvent extraction of the organic acid products should ideally have at least some of the following characteristics:

little to no miscibility with water;

little to no miscibility with the mineral acid;

selectively partition the organic acids into the organic solvent phase;

have low partitioning of the mineral acid into the organic solvent phase;

have low reactivity between the organic extraction solvent and the mineral acid;

have low reactivity between the organic extraction solvent & the organic acid products;

have the ability to remove or reduce any mineral acid that partitions into the organic phase;

easy to remove from organic acid, such as by backwashing or distillation;

allow the neutralization the organic acids.

In one embodiment, the partition coefficient of the extraction solvent for levulinic acid is at least 0.3, more specifically, at least 0.5, more specifically, at least 0.7, more specifically, at least 1.0, more specifically at least 1.3, more specifically, at least 1.5 more specifically, at least 1.7, and more specifically at least 2.0. In one embodiment, the partition coefficient of the extraction solvent for formic acid is at least 0.3, more specifically, at least 0.5, more specifically, at least 0.7, more specifically, at least 1.0, more specifically at least 1.3, more specifically, at least 1.5 more specifically, at least 1.7, and more specifically at least 2.0, more specifically, at least 2.3, more specifically, at least 2.5, more specifically, at least 3.0, more specifically, at least 3.5, more specifically, at least 4.0, more specifically, at least 5.0 more specifically, at least 6.0, more specifically, at least 7.0, more specifically, at least 8.0, and more specifically, at least 9.0.

In one embodiment, to conduct a CSTR reaction with a given “residence time” t (in this case, t=typically 30 min to 1 hour) the volume of the reactor is selected such that the typical “residence time” of the reactants is the designed target. The mass of material held in the reactor is designed to be the product of the mass flow rate into the reactor and the residence time. Longer residence time=larger quantity of material held in the reactor. Slower feed rate=smaller quantity of material held in the reactor. In operation, it is desirable for the feed to be a constant flow rate and composition; also the exit stream is a constant flow rate and composition, and the sum of the flow rates of all exit streams equals the flow rate of the feeds (on a mass basis).

Typically, the reactor goes through a start-up phase until the reactor achieves “steady state” wherein the reactor contents, temperature, and pressure only varies within a controlled range. After steady state is achieved, the reactor is continuously operated as long as desired (days, weeks, months, years). During operation, the feed is steady, and the exit stream is steady. The reactor contents are steady. But the average residence time of the reactor contents is designed and held constant. The reactor content composition is equal to the composition of the exit streams.

During the startup phase, many strategies can be used to reach steady state as quickly as possible. For example, the reactor contents may be started as 100% water, or fed with the desired steady state composition of the reactor contents. The composition of the feed streams can be allowed to vary, and the flow rate of the exit stream may be varied to achieve steady state (anywhere from zero to equal to the feed rate).

It has been observed that the production of HMF could potentially lead to large amounts of undesirable char build up. For example, a CSTR design which is inadvertently designed so as to run at conditions which give a high HMF yield, could be expected to yield high char and discouraging results.

It is thus, one technical advantage of one embodiment of the invention to provide a continuous reaction system in such a way to minimize the HMF concentration.

It has been observed in a batch reaction wherein the HMF concentration starts out at zero, builds to a peak, and then declines again to very low levels. In a simple batch reaction, such a profile is difficult to avoid. Likewise, a single, continuous, plug-flow reactor could experience a similar HMF concentration along the length of the tube. The inventors have found that in one embodiment, a carefully designed reaction system (for example, an initial CSTR followed by a plug flow reactor) could avoid having a high HMF concentration and still achieve high conversion.

The following paragraphs provide for various aspects of the present invention. In one embodiment, in a first paragraph (1), the present invention provides a method to prepare levulinic acid comprising the steps:

a) heating a first mixture comprising water and sulfuric acid to about 80° C. to about 160° C. in a reactor; and

b) adding a second mixture of water and a sugar selected from glucose and sucrose to the heated solution over a first period of time to form a liquid reaction mixture in a reactor, wherein the liquid reaction mixture comprises between 20% and 60% sulfuric acid to form a reaction mixture including levulinic acid; and

c) recovering levulinic acid.

2. The method of paragraph 1, wherein the first period of time is between 10 and 300 minutes.

3. The method of paragraph 1, wherein the first period of time is between 20 and 240 minutes.

4. The method of paragraph 1, wherein the first period of time is between 30 and 180 minutes.

5. The method of paragraph 1, wherein the first period of time is between 60 and 180 minutes.

6. The method of paragraph 1, wherein the first period of time is between 60 and 120 minutes.

7. The method of any of paragraphs 1 through 6, further comprising the step of heating the reaction mixture for a reaction period at a first reaction temperature after all of the second mixture has been added to the reactor.

8. The method of paragraph 7, wherein the reaction period is between 10 and 300 minutes.

9. The method of paragraph 7, wherein the reaction period is between 20 and 240 minutes.

10. The method of paragraph 7, wherein the reaction period is between 30 and 180 minutes.

11. The method of paragraph 7, wherein the reaction period is between 60 and 180 minutes.

12. The method of paragraph 7, wherein the reaction period is between 60 and 120 minutes.

13. The method of any of paragraphs 7 through 12, wherein the first reaction temperature is between approximately 100 to about 180° C.

14. The method of paragraph 13, wherein the first reaction temperature is between approximately 100 to about 160° C.

15. The method of paragraph 13, wherein the first reaction temperature is between approximately 100 to about 140° C.

16. The method of paragraph 13, wherein the first reaction temperature is between approximately 120 to about 140° C.

17. The method of any of paragraphs 1 through 16, further comprising the steps:

subjecting the reaction mixture to an extraction solvent to extract levulinic acid into an extract phase;

removing the extract phase from the reaction mixture; and

recovering the levulinic acid from the extract phase.

17a. The method of paragraph 17, wherein the extraction solvent is a phenol.

18. The method of paragraph 17a, wherein the phenol is a halogenated phenol.

19. The method of paragraph 17a, wherein the phenol is an alkyl phenol.

20. The method of paragraph 19, wherein the alkyl phenol is xylenol.

21. The method of paragraph 19, wherein the alkyl phenol is a mixture of xylenol isomers.

21a. The method of any of paragraphs 17 through 21, wherein recovering levulinic acid from the extract phase comprises distillation or crystallization.

21b. The method of any of paragraphs 17 through 21, wherein recovering levulinic acid from the extract phase comprises esterification with an alkanol to create a levulinic ester, followed by distillation of the levulinic ester.

21c. The method of any of paragraphs 17 through 21b, further comprising recovering formic acid from the extract phase.

21d. The method of paragraph 21c, wherein recovering formic acid from the extract phase comprises distillation or crystallization.

21e. The method of paragraph 21c, wherein recovering formic acid from the extract phase comprises esterification with an alkanol to create a formic ester, followed by distillation of the formic ester.

22. The method of any of paragraphs 1 through 16, further comprising the steps:

filtering solids from the reaction mixture, optionally after cooling;

adding a water immiscible liquid to the reaction mixture so that thereaction mixture forms first and second layers, wherein greater than 90% of the sulfuric acid is in the first layer and greater than 90% of the water immiscible liquid is in the second layer;

recovering levulinic acid and optionally formic acid from the second layer; and

recycling the first layer back to the reactor.

23. The method of paragraph 22, wherein the water immiscible liquid is a phenol.

24. The method of paragraph 23, wherein the phenol is a halogenated phenol.

25. The method of paragraph 23, wherein the phenol is an alkyl phenol.

26. The method of paragraph 25, wherein the alkyl phenol is xylenol.

27. The method of paragraph 22, wherein the alkyl phenol is a mixture of xylenol isomers.

27a. The method of any of paragraphs 22 through 27, wherein recovering levulinic acid or formic acid from the second layer comprises distillation or crystallization.

27b. The method of any of paragraphs 22 through 27, wherein recovering levulinic acid from the second layer comprises esterification with an alkanol to create a levulinic ester, followed by distillation of the levulinic ester.

27c. The method of any of paragraphs 22 through 27, wherein recovering formic acid from the second layer comprises esterification with an alkanol to create a formic ester, followed by distillation of the formic ester.

28. The method of any of paragraphs 1 through 27, wherein the levulinic acid is recovered in a yield greater than 40% mol.

29. The method of any of paragraphs 1 through 27, wherein the levulinic acid is recovered in a yield greater than 45% mol.

30. The method of any of paragraphs 1 through 27, wherein the levulinic acid is recovered in a yield greater than 50% mol.

31. The method of any of paragraphs 1 through 30 wherein the second mixture is water and glucose.

32. The method of paragraph 31, wherein the greater than 75% of the glucose is converted.

33. The method of paragraph 31, wherein the greater than 80% of the glucose is converted.

34. The method of any of paragraphs 31, wherein the greater than 85% of the glucose is converted.

35. The method of any of paragraphs 1 through 30 wherein the second mixture is water and sucrose.

36. The method of claim any of paragraphs 1 through 35, wherein the reactor is a continuous addition batch reactor.

36a. The method of claim any of paragraphs 1 through 35, wherein the reactor is a CSTR reactor.

37. The method of paragraph 35, wherein the reactor is a multi stage reactor comprising at least a first reactor and a second reactor.

38. A method to prepare levulinic acid comprising the steps:

heating a first mixture comprising water and sulfuric acid to about 80 to about 160° C. to form a solution;

adding a second mixture of sugar and water to the heated solution over a first period of time to form a liquid reaction mixture in a first reactor, wherein the liquid reaction mixture comprises between 20% and 60% sulfuric acid;

heating the reaction mixture for a first reaction period at a first reaction temperature after all of the second mixture has been added to the first reactor;

feeding the liquid reaction mixture to a second reactor;

heating the reaction mixture for a second reaction period at a second reaction temperature after all of the second mixture has been added to the second reactor; and

recovering levulinic acid.

39. The method of paragraph 38, further comprising the step of recirculating the reaction mixture from the second reactor to the first reactor.

40. The method of paragraphs 38 or 39, further comprising the step of adding a third mixture comprising a solution of sugar and water to the second reactor.

41. The method of paragraph 40, wherein the addition of the third mixture to the second reactor is simultaneous with the addition of the second mixture to the first reactor.

42. The method of any of paragraphs 38 through 41, wherein the sugar is selected from the group consisting of fructose, glucose, sucrose and mixtures thereof.

43. The method of paragraph 42, wherein the sugar is sucrose.

44. The method of any of paragraphs 38 through 43, wherein the first period of time is between 10 and 300 minutes.

45. The method of any of paragraphs 38 through 43, wherein the first period of time is between 20 and 240 minutes.

46. The method of any of paragraphs 38 through 43, wherein the first period of time is between 30 and 180 minutes.

47. The method of any of paragraphs 38 through 43, wherein the first period of time is between 60 and 180 minutes.

48. The method of any of paragraphs 38 through 43, wherein the first period of time is between 60 and 120 minutes.

49. The method any of paragraphs 38 through 48, wherein the first reaction period is between 10 and 300 minutes.

50. The method of any of paragraphs 38 through 48, wherein the first reaction period is between 20 and 240 minutes.

51. The method of any of paragraphs 38 through 48, wherein the first reaction period is between 30 and 180 minutes.

52. The method of any of paragraphs 38 through 48, wherein the first reaction period is between 60 and 180 minutes.

53. The method of any of paragraphs 38 through 48, wherein the first reaction period is between 60 and 120 minutes.

54. The method of any of paragraphs 38 through 53, wherein the first reaction temperature is between approximately 100 to about 180° C.

55. The method of any of paragraphs 38 through 53, wherein the first reaction temperature is between approximately 90 to about 130° C.

56. The method of any of paragraphs 38 through 53, wherein the first reaction temperature is between approximately 100 to about 130° C.

57. The method of any of paragraphs 38 through 53, wherein the first reaction temperature is between approximately 110 to about 120° C.

58. The method of any of paragraphs any of claims 38 through 57, wherein the second reaction period is between 10 and 300 minutes.

59. The method of any of paragraphs 38 through 57, wherein the second reaction period is between 20 and 240 minutes.

60. The method of any of paragraphs 38 through 57, wherein the second reaction period is between 30 and 180 minutes.

61. The method of any of paragraphs 38 through 57, wherein the second reaction period is between 60 and 180 minutes.

62. The method of any of paragraphs 38 through 57, wherein the first reaction period is between 60 and 120 minutes.

63. The method of any of paragraphs 38 through 62, wherein the second reaction temperature is between approximately 120 to about 180° C.

64. The method of any of paragraphs 38 through 62, wherein the second reaction temperature is between approximately 120 to about 160° C.

65. The method of any of paragraphs 38 through 62, wherein the second reaction temperature is between approximately 130 to about 150° C.

66. The method of any of paragraphs 38 through 62, wherein the second reaction temperature is between approximately 130 to about 140° C.

67. The method of any of paragraphs 38 through 66, further comprising the steps:

subjecting the reaction mixture to an extract solvent to extract levulinic acid into an extract phase;

removing the extract phase from the reaction mixture; and

recovering the levulinic acid from the extract phase.

68. The method of paragraph 67, wherein the extract solvent is a phenol.

69. The method of paragraph 68, wherein the phenol is a halogenated phenol.

70. The method of paragraph 68, wherein the phenol is an alkyl phenol.

71. The method of paragraph 70, wherein the alkyl phenol is xylenol.

71. The method of paragraph 70, wherein the alkyl phenol is a mixture of xylenol isomers.

72. The method of any of paragraphs 67 through 71, wherein recovering levulinic acid from the extract phase comprises distillation or crystallization.

73. The method of any of paragraphs 67 through 71, wherein recovering levulinic acid from the extract phase comprises esterification with an alkanol to create a levulinic ester, followed by distillation of the levulinic ester.

74. The method of any of paragraphs 67 through 73, further comprising recovering formic acid from the extract phase.

75. The method of paragraph 74, wherein recovering formic acid from the extract phase comprises distillation or crystallization.

76. The method of paragraph 74, wherein recovering formic acid from the extract phase comprises esterification with an alkanol to create a formic ester, followed by distillation of the formic ester.

77. The method of any of paragraphs 38 through 66, further comprising the steps:

filtering solids from the reaction mixture, optionally after cooling;

adding a water immiscible liquid to the reaction mixture so that the reaction mixture forms first and second layers, wherein greater than 90% of the sulfuric acid is in the first layer and greater than 90% of the water immiscible liquid is in the second layer;

recovering levulinic acid and optionally formic acid from the second layer; and

recycling the first layer back to the first or second reactor.

78. The method of paragraph 77, wherein the water immiscible liquid is a phenol.

79. The method of paragraph 78, wherein the phenol is a halogenated phenol.

80. The method of paragraph 78, wherein the phenol is an alkyl phenol.

81. The method of paragraph 80, wherein the alkyl phenol is xylenol.

82. The method of paragraph 80, wherein the alkyl phenol is a mixture of xylenol isomers.

83. The method of any of paragraphs 77 through 82, wherein recovering levulinic acid or formic acid from the second layer comprises distillation or crystallization.

84. The method of any of paragraphs 77 through 82, wherein recovering levulinic acid from the second layer comprises esterification with an alkanol to create a levulinic ester, followed by distillation of the levulinic ester.

85. The method of any of paragraphs 77 through 84, wherein recovering formic acid from the second layer comprises esterification with an alkanol to create a formic ester, followed by distillation of the formic ester.

In any of the above embodiments, the sugar can be glucose. Additionally, the ratio of glucose to water in the second mixture can be anywhere between 0.1:99.9 to 99.9:0.1 by weight, more specifically, between 1:99 to 99:1, more specifically, between 5:95 to 95:5, more specifically, between 10:90 to 90:10, more specifically, between 20:80 to 80:20, more specifically, between 30:70 to 70:30, more specifically, between 40:60 to 60:40, more specifically, between 45:65 to 65:45, and more specifically, the ratio is approximately 50:50 by weight.

In any of the above embodiments, the sugar can be sucrose. Additionally, the ratio of sucrose to water in the second mixture can be anywhere between 0.1:99.9 to 99.9:0.1 by weight, more specifically, between 1:99 to 99:1, more specifically, between 5:95 to 95:5, more specifically, between 10:90 to 90:10, more specifically, between 20:80 to 80:20, more specifically, between 30:70 to 70:30, more specifically, between 40:60 to 60:40, more specifically, between 45:65 to 65:45, and more specifically, the ratio is approximately 50:50 by weight.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

In one aspect, the invention is directed to a process to make crystallizable levulinic acid (“LA”) from sugar solutions.

Hydrolysis of a 1-3 Molar solution of sucrose, glucose, fructose, or blends of the aforementioned, specifically fructose and sucrose, occurs in a batch or continuous reactor, specifically a continuous reactor. In one embodiment the method includes the following steps following hydrolysis of a 1-3 Molar solution of sucrose, glucose, fructose, or blends of the aforementioned:

(a) Filtration of solids from hydrolysate mixture.

(b) Water or extraction solvent wash of solids (optional).

(c) Extraction of LA and formic acid from aqueous hydrolysate into an extraction solvent.

(d) Removal of extraction solvent by distillation.

(e) Thin-film evaporation of LA.

(f) Crystallization of LA.

(g) recovery of formic acid.

The process allows fast reaction time, easy to handle char byproduct, good yields, no neutralization step (optional), efficient extraction and distillation to afford a crystallizable LA product.

A few processes are known to make LA from sugar, but little is known on how to remove the LA and formic acid from the reactor and purify it from the hydrolysate. The disclosed process produces approximately 97% purity LA that crystallizes.

LA can be further converted into various useful esters. One such method includes reactive distillation. Such a process includes introducing a carboxylic acid and an alcohol into a reaction column. The bottom stream, for example, comprises the ester formed and unreacted carboxylic acid. The overhead stream comprises unreacted alcohol and water. The reactants can then be recycled for additional reactive distillation.

It should be appreciated that reactive distillation processes generally don't have “steps”. That is the reaction/distillation/conversion all take place in a reaction zone and there is no true sequence of step. In one embodiment, a reactive distillation process includes feeding levulinic acid, water, and a monohydroxy alcohol into a distillation column, wherein a heterogeneous catalyst is suspended in one or more stages. Generally, the distillation column is heated from the bottom and has a reflux flow to effect separation of the levulinic ester from the mixture and byproducts. In another embodiment, for a homogeneous catalyst reactive distillation, the reactive distillation process includes feeding levulinic acid, water, a monohydroxy alcohol and a homogeneous catalyst into a distillation column. The distillation column is heated (e.g., from the bottom) and has a reflux flow to effect separation of the levulinic ester from the mixture and byproducts.

For example, levulinic acid, water and a monohydroxy alcohol along with an optional acid catalyst can be combined to form a mixture. The mixture can be heated in a reactive distillation column with a heterogeneous acid catalyst to effect esterification of the levulinic acid to afford the levulinic ester. The levulinic ester is separated from the mixture, starting materials, and byproducts via a subsequent purification process. It is advantageous to remove metal ions from the reaction mixture components prior to the reactive distillation process to prevent neutralization of the heterogenous acid catalyst and to prevent unwanted side reactions that could form undesired byproducts, such as lactones. In one embodiment, higher molecular weight oliogomers and extraction solvents (such as substituted phenols, xylenols, cresols, etc.) are removed from the stream using activated carbon prior to reactive distillation. In another embodiment, higher molecular weight oliogomers and extraction solvents (such as substituted phenols, xylenols, cresols, etc.) are removed from the stream using activated carbon subsequent to reactive distillation. In another embodiment, sulfuric acid is removed from the stream by anion exchange resins, basic alumina (powder or bead), weak bases, or molecular sieves prior to reactive distillation. In another embodiment, sulfuric acid is removed from the stream by anion exchange resins, weak bases, or molecular sieves subsequent to reactive distillation. These embodiments are useful because the higher molecular weight oligomers could foul the heterogeneous acid catalyst. Also, the extraction solvent could undergo acid catalyzed side reactions with LA or LA esters. Additionally, the sulfuric acid impurities could catalyze unwanted side reactions of LA and LA esters. The reactive distillation of acids and alcohols are known to those of skill in the art.

The following paragraphs also provide for further still additional aspects of the present invention. In one embodiment, in a first paragraph (1) a reactive distillation process is described by combining levulinic acid, water and a monohydroxy alcohol in a reactive distillation column comprising a suspended catalyst to form a mixture; heating the mixture in the reactive distillation column to effect esterification of the levulinic acid to afford the levulinic ester; and separating the levulinic ester from the mixture and byproducts, wherein removal of metal ions from the reaction mixture components is effected prior to and/or after the reactive distillation process.

2. The reactive distillation process of paragraph 1, wherein the metal ions are removed prior to the reactive distillation process by cation exchange resins.

3. A reactive distillation process is described by combining levulinic acid, water and a monohydroxy alcohol alcohol in a reactive distillation column comprising a suspended catalyst to form a mixture; heating the mixture in a reactive distillation column to effect esterification of the levulinic acid to afford the levulinic ester; and separating the levulinic ester from the mixture and byproducts, wherein oligomers and solvents are removed by adsorption or adsorption via carbon bed from the reaction mixture components prior to and/or after the reactive distillation process.

4 A reactive distillation process is described by combining levulinic acid, water and a monohydroxy alcohol in a reactive distillation column comprising a suspended catalyst to form a mixture; heating the mixture in a reactive distillation column to effect esterification of the levulinic acid to afford the levulinic ester; and separating the levulinic ester from the mixture and byproducts, wherein sulfuric acid impurities are removed by anion exchange resins, weak bases, or molecular sieves from the reaction mixture components prior to and/or after the reactive distillation process.

5. The reactive distillation process of any of paragraphs 1 through 4, comprising a further step of treating the levulinic acid or ester with molecular sieves or an alkali or alkaline metal base to remove any sulfuric acid impurities from the levulinic ester.

6. The reactive distillation process of any of paragraphs 1 through 5, further comprising the step of washing the levulinic acid with water prior to reactive distillation and/or washing the levulinic ester with water, to remove water soluble impurities, such as sulfuric acid.

7. The reactive distillation of any of paragraphs 1 through 7, further comprising the step of removing alpha and beta angelica lactone and/or a 4-alkoxy-gamma valerolactone impurities by distillation or by conversion to other compounds followed by distillation, or by adsorption with a suitable absorption media.

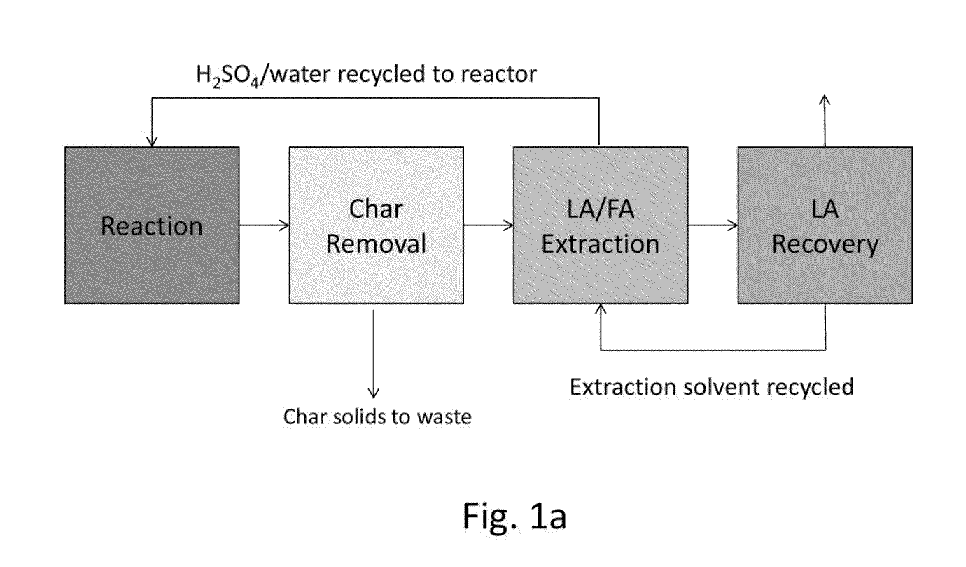

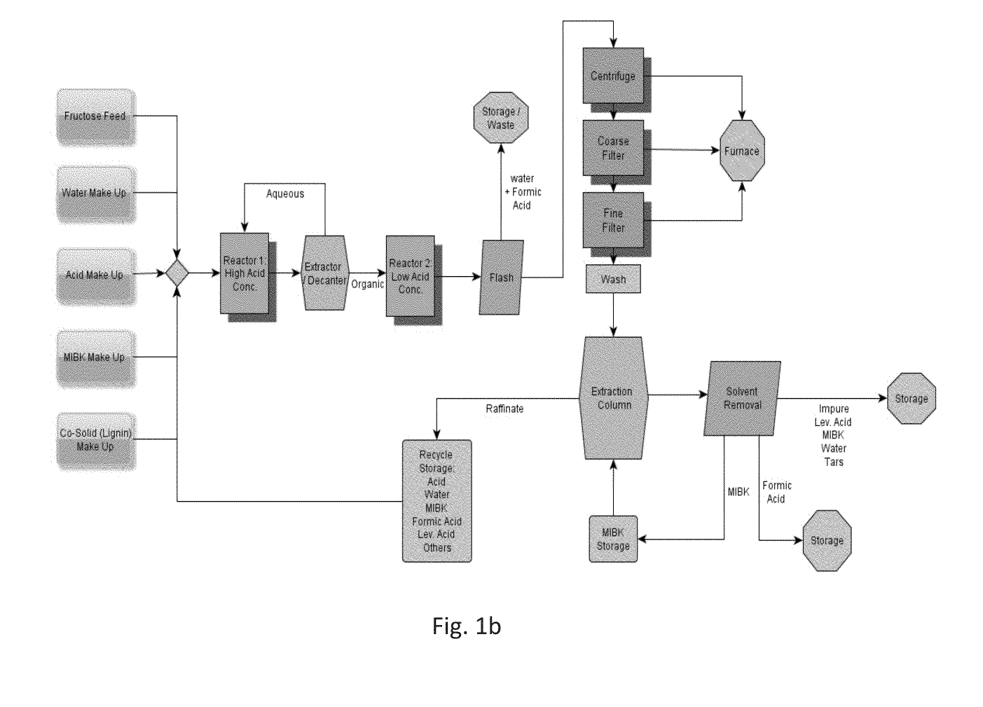

Referring now to FIGS. 1a and 1b. FIG. 1a provides a general process description for one embodiment for the production of levulinic acid. Water, mineral acid and biomass are added to a reactor under reaction conditions to convert the biomass into various products, including levulinic acid and formic acid as well as solids char. The solids are then removed from the reaction mixture. The reaction mixture is then combined with an extraction solvent, which extracts a majority of the levulinic acid and formic acid from the water and sulfuric acid. In one embodiment, the formic acid is removed from the hydrolysate, or reaction mixture, either before or after the solids removal step but prior to adding the extraction solvent for levulinic acid. This can be accomplished by methods known in the art, such as distillation, steam stripping or extraction. In other embodiments, the formic acid can be extracted out of the reaction mixture after the extraction of levulinic acid utilizing a different extraction solvent than that used for levulinic acid. In still another embodiment, the formic acid and levulinic acid are both extracted using the same extraction solvent. The water and sulfuric acid is then optionally recycled back to the reactor and the formic acid and levulinic acid are separated from the extraction solvent, after which the extraction solvent can be recycled back to be re-used in the extraction step.

The reactor can be a batch reactor, a CSTR or a plug reactor. The mineral acid is sulfuric acid (H₂SO₄), hydrochloric acid (HCl), hydrobromic acid (HBr) or hydroiodic acid (HI), preferably sulfuric acid. The biomass comprises sludges from paper manufacturing process; agricultural residues; bagasse pity; bagasse; molasses; aqueous oak wood extracts; rice hull; oats residues; wood sugar slops; fir sawdust; naphtha; corncob furfural residue; cotton balls; raw wood flour; rice; straw; soybean skin; soybean oil residue; corn husks; cotton stems; cottonseed hulls; starch; potatoes; sweet potatoes; lactose; sunflower seed husks; sugar; corn syrup; hemp; waste paper; wastepaper fibers; sawdust; wood; residue from agriculture or forestry; organic components of municipal and industrial wastes; waste plant materials from hard wood or beech bark; fiberboard industry waste water; post-fermentation liquor; furfural still residues; and combinations thereof, a C5 sugar, a C6 sugar, a lignocelluloses, cellulose, starch, a polysaccharide, a disaccharide, a monosaccharide or mixtures thereof. Preferably the biomass is high fructose corn syrup, a mixture of at least two different sugars, sucrose, an aqueous mixture comprising fructose, an aqueous mixture comprising fructose and glucose, an aqueous mixture comprising hydroxymethylfurfural, an aqueous solution of fructose and hydroxymethylfurfural, an aqueous mixture of glucose, an aqueous mixture of maltose, an aqueous mixture of inulin, an aqueous mixture of polysaccharides, or mixtures thereof, and more preferably, the biomass comprises fructose, glucose or a combination thereof.

FIG. 1b provides a more specific process description for one embodiment for the production of levulinic acid.

Feeds

Concentration of feeds are controlled to maintain desired reaction stoichiometry. “Make-up” stream flows are controlled based on the composition and flow rate of the recycle stream.

Reactors

One, optionally two, reactors are used to convert the sugars, specifically, fructose, glucose or sucrose to the desired products. The reactors are optionally vented to maintain an internal pressure; the vent stream is optionally collected to recover steam and formic acid product; the vent stream can all be returned to the reactor as a reflux. If there are two reactors in series, the first reactor is optionally controlled at a different temperature and at a high concentration of acid in order to achieve desired conversion and selectivity. The first reactor would generally be controlled at a lower temperature than the second. Optionally, a process step between the two reactors may be used to separate “tar” solids and/or to preferentially extract the reaction products (away from the aqueous feed) to feed into the second reactor.

The reactors may be operated in a batch-wise (wherein the reactants are fed to the reactor and the reaction continues until the desired degree of conversion, and the products are then emptied from the reactor) or in a continuous fashion (wherein reactants are fed continuously and the products are removed continuously). In one embodiment, the reactors are run in a continuous fashion with products removed in a steady fashion or the reactants are removed in a pulsed fashion. In another embodiment, the reactors are run in a batch mode, with the biomass preferably being added to the reactor over a period of time t.

The agitation in the reactors should be adequate to prevent agglomeration of solid co-products which may be formed during the reaction. Specifically, the reactors should be designed with sufficient axial flow (from the center of the reactor to the outer diameter and back).

Flash

The reaction products may be optionally cooled in a “flash” process. The flash step rapidly cools the reaction products by maintaining a pressure low enough to evaporate a significant fraction of the products. This pressure may be at or below atmospheric pressure. The evaporated product stream may be refluxed through stages of a distillation column to minimize the loss of desired reaction products, specifically levulinic acid, and to ensure recovery of formic acid reaction products and solvent. Recovered solvent may be recycled back to reactor 1 or 2.

The “bottoms” or less volatile stream from the flash step is advanced to the solids separation stage.

Solids Separation

In the solids separation stage of the process, the solvent and desired reaction products, specifically levulinic acid and formic acid, are separated from any solids which may have formed during the reaction phase. The solids may be separated through a combination of centrifuge, filtration, and settling steps (ref Perrys Chemical Engineering Handbook, Solids Separation). The separated solids may be optionally washed with water and solvents to recover desired reaction products or solvent which may be entrained in or adsorbed to the solids. It has been found that in some embodiments, such as those reactions employing high levels of mineral acid (greater than 20%) that are reacted at lower temperatures, such as between 60-110 C, the solids may have density properties similar to the liquid hydrolysate which effectively allows the solids to be suspended in solution. In these embodiments, certain separation techniques such as centrifugation are not as effective. In these embodiments filtration utilizing filter media having a pore size less than about 20 microns has been found to effectively remove solids from the mixture. When removing solids from the system a solid “cake” is formed. It is desirable that the cake be up to 50% solids. Thus any separation technique that obtains a cake having a higher amount of solids is preferred. A certain amount of LA and mineral acid will be present in the cake and it may be desirable to wash the cake with an extraction solvent or water to recover LA.

It has also been surprisingly found that the solid particles in the high mineral acid and lower temperature embodiments are easily filtered and do not inhibit flow as the cake is formed. It is believed that the properties of the char formed under these process conditions are such that any cake remains porous enough that a small filter size (less than 20 microns) can be utilized while maintaining a high flow rate through the medium.

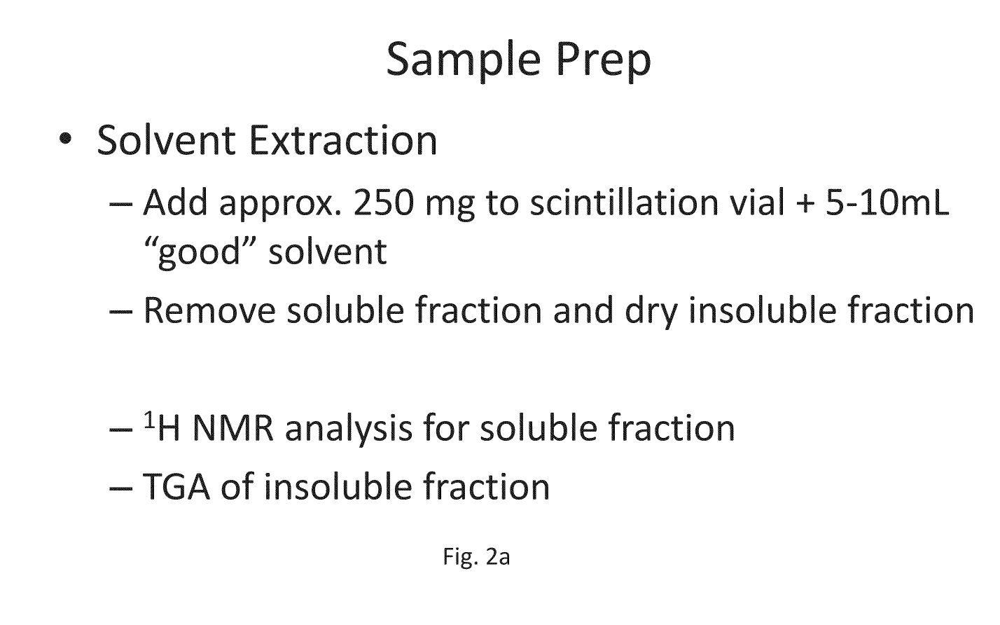

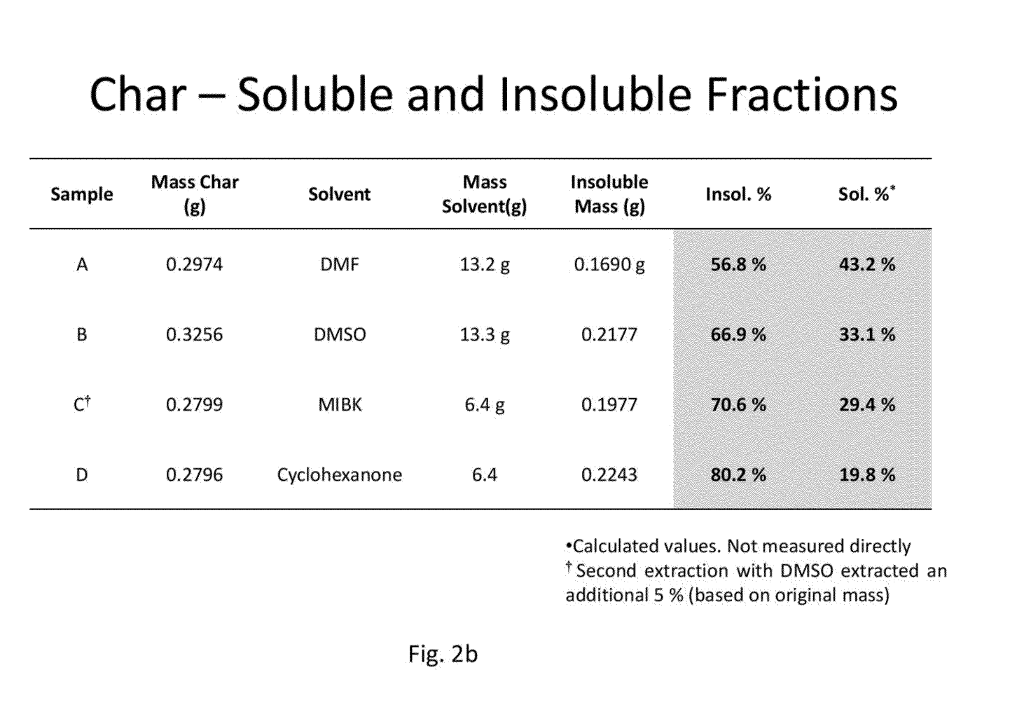

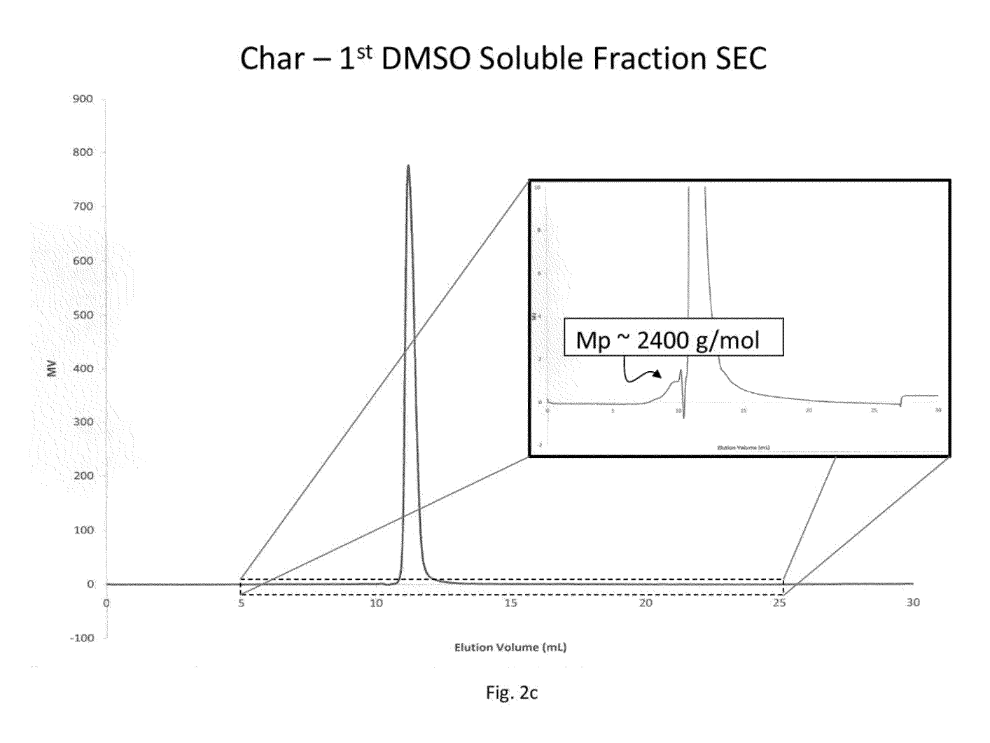

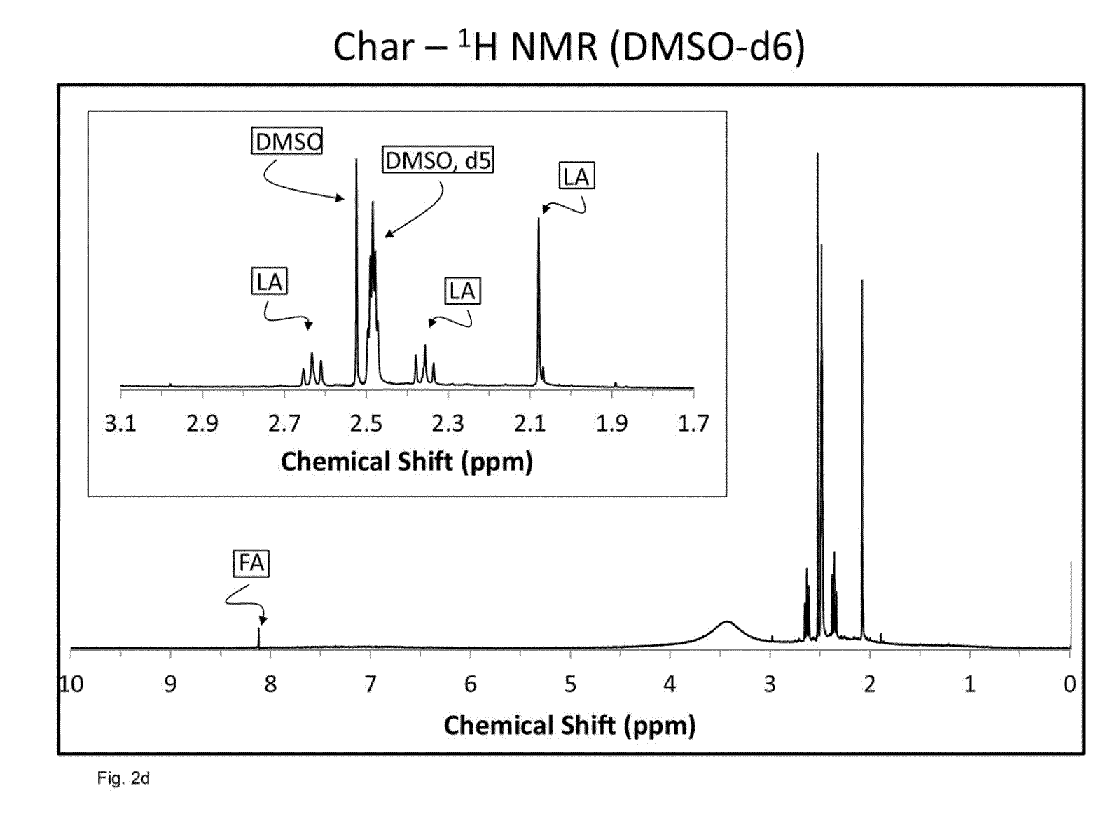

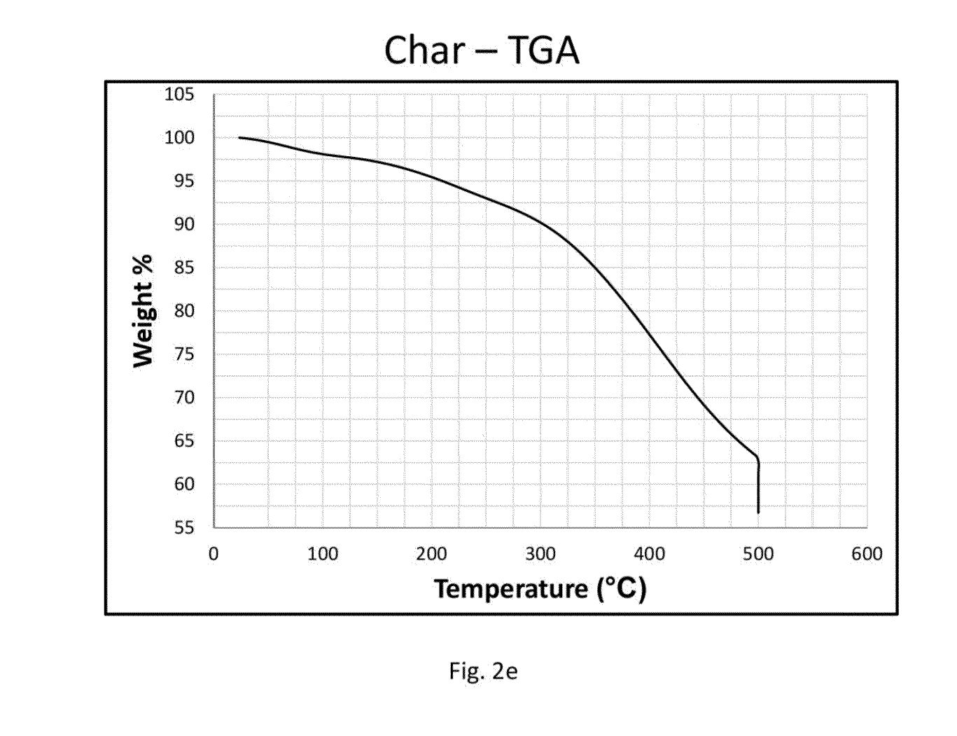

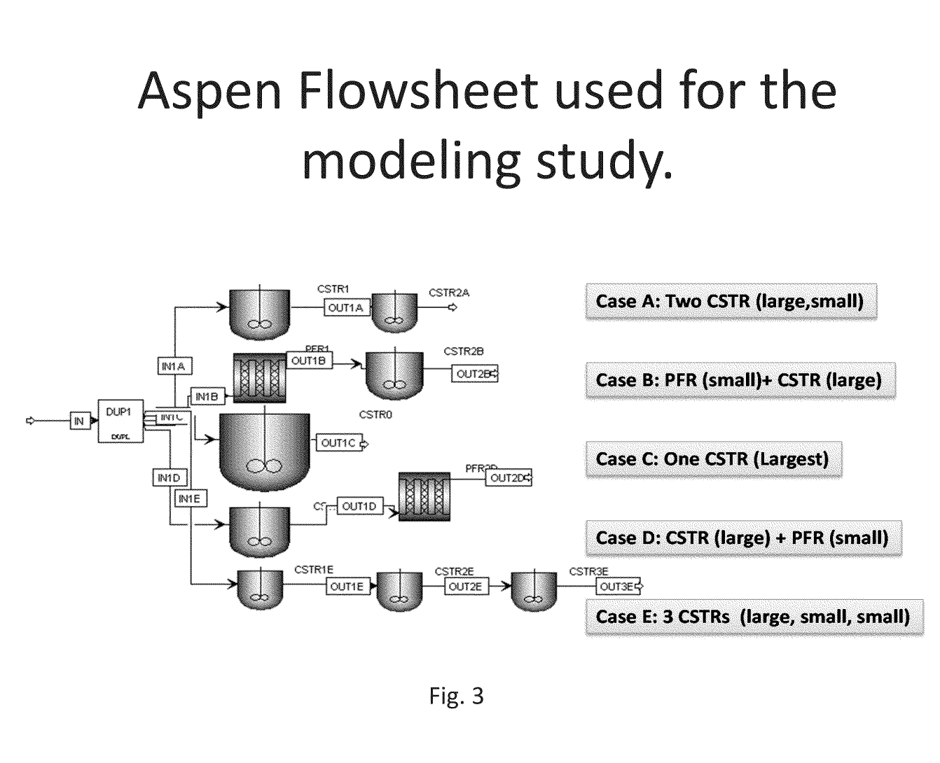

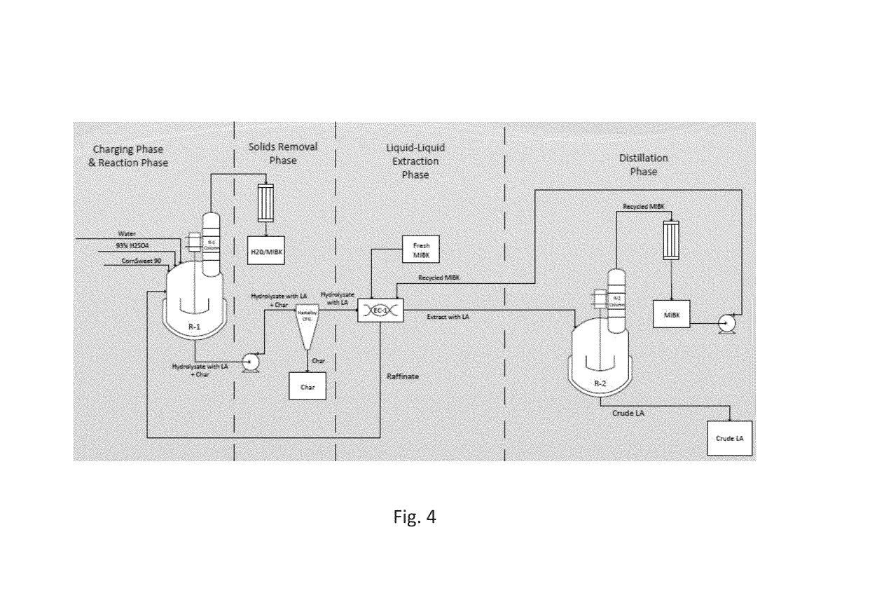

Referring now to FIGS. 2a through 2e, solid, black char was isolated from a fructose hydrolysate reaction mixture by filtration. The char was rinsed with water 2 times to recover additional levulinic acid and formic acid, and then, the char was dried at 50-60° C. and 30 Torr for at least 12 h. The dried char was subjected to solvent extraction according to FIG. 2b. A considerable amount of material was extracted from the char. Proton NMR was used to analyze the soluble extract fraction, and it was found to contain mostly levulinic acid and formic acid. Thus, this solvent extraction method is surprisingly advantageous for further recovery of levulinic acid from the reaction mixture.

The isolated solids may be incinerated to generate power or disposed.

The liquid stream, comprising (but not limited to) water, acid, solvent, levulinic acid, formic acid, and some “soluble tars” are advanced to the extraction stage of the process.

Extraction

In the extraction stage of the process, the liquid stream is mixed with an extraction solvent stream. The preferred extraction solvent dissolves levulinic acid more effectively than the other products in the liquid stream. The preferred solvent does not dissolve significantly into the water phase. Extraction configurations are preferably multi-stage and continuous, as described in Perry's Chemical Engineering Handbook.

The aqueous raffinate is recycled to the reactor phase, after optional distillation or purification steps to adjust the relative concentrations of solvent, water, and acid in the raffinate.

The extract solvent phase contains levulinic acid and formic acid and is progressed to the solvent removal stage of the process.

Suitable solvents to extract LA include, for example, polar water-insoluble solvents such as MIBK, MIAK, cyclohexanone, o, m, and para-cresol, xylenol, chlorinated phenols, substituted phenols, for example, 2-sec butyl phenol, C4-C18 alcohols, such as n-pentanol, isoamyl alcohol, n-heptanol, 2-ethyl hexanol, n-octanol, 1-nonanol, cyclohexanol, methylene chloride, 1,2-dibutoxy-ethylene glycol, acetophenone, isophorone, o-methoxy-phenol, methyl-tetrahydrofuran, tri-alkylphosphine oxides (C4-C18) and ortho-dichlorobenzene and mixtures thereof. Such solvents are used generally at room temperature so as not to serve as potential reaction component.

Solvent Removal

Levulinic acid may be separated from the solvent phase by evaporating or distilling the solvent. Alternatively, the levulinic acid may be crystallized from the solvent phase in a crystallization process. The solvent removal process may be a combination of distillation and crystallization. The recovered solvent may be recycled to the extraction step or to the reactor step.

The resulting stream of highly concentrated levulinic acid may be advanced for further chemical derivatization or may be further purified in another distillation step such as high vacuum wipe-film-evaporation or falling film evaporation. Preferably the levulinic acid stream is kept at a low temperature throughout the solvent removal steps to inhibit the formation of angelica lactone.

Mineral Acids

Suitable acids used to convert the biomass materials described herein, including sugars, include mineral acids, such as but not limited, to sulfuric acid, hydrochloric acid, hydrobromic acid, hydroiodic acid, nitric acid, phosphoric acid, boric acid, hydrofluoric acid, perchloric acid and mixtures thereof.

### EXAMPLES

### Examples 1-12

375 g of a 64 wt % H₂ SO₄ solution was charged to a Zricodyne 702 Parr along with 81 g of deionized water. The reactor was sealed and agitation rate set to approximately 100 RPM before pressure testing and pre-heating the system to the desired reaction temperature. When the reactor reached the desired temperature, 144 g of a 50 wt % solution of glucose in water was fed into the reactor over the time period listed below in Table 1. After all the glucose had been fed, the reactor was held at the reaction temperature for an additional 60 minutes before cooling the reactor in an ice bath to room temperature for analysis. The sample was then filtered through a 1.1 μm glass mat filter to remove any solid particulate. The solids were dried at 125° C. to constant mass to obtain an accurate weight. Upon filtration, it was observed that the solid particulates did not did not adhere to the reactor internals, including the agitator and reactor side walls. The filtrate was analyzed by HPLC on a Bio-Rad Aminex column using a mobile phase consisting of 20 mM phosphoric acid augmented with 3% acetonitrile. The initial and final reactor composition of examples 1-12 are summarized in Table 1:

TABLE 1

| | | | | Feed | | | | | | | | | | |
|---|---|---|---|---|---|---|---|---|---|---|---|---|---|---|
| | % | % | Temp | Time | Cook Time | | | | LA wt | LA mol % | FA mol | LA Molar | Glucose | LA/ |
| Example | H2SO4 | Glucose | (° C.) | (min) | (min) | % LA | % FA | % Glu | % Yield | Yield | % yield | Selectivity | Conversion | FA |
| 1 | 40% | 15% | 120 | 65 | 60 | 4.51 | 1.94 | 1.72 | 29.75 | 46.49 | 50.51 | 52.50% | 88.54% | 0.92 |
| 2 | 40% | 15% | 130 | 60 | 60 | 5.35 | 2.07 | 0.42 | 35.28 | 55.12 | 53.67 | 56.71% | 97.20% | 1.03 |
| 3 | 45% | 15% | 120 | 60 | 60 | 4.74 | 2.41 | 0.63 | 31.39 | 49.05 | 62.80 | 51.20% | 95.78% | 0.78 |
| 4 | 45% | 15% | 130 | 60 | 60 | 4.42 | 1.86 | 0.36 | 29.28 | 45.75 | 48.75 | 46.89% | 97.57% | 0.94 |
| 5 | 40% | 15% | 120 | 120 | 60 | 4.86 | 2.05 | 1.09 | 32.15 | 50.24 | 53.34 | 54.15% | 92.76% | 0.94 |
| 6 | 45% | 15% | 120 | 120 | 60 | 4.81 | 1.76 | 0.84 | 31.92 | 49.88 | 45.92 | 52.83% | 94.41% | 1.09 |
| 7 | 45% | 10% | 120 | 60 | 60 | 3.16 | 1.60 | 0.63 | 31.22 | 48.78 | 62.48 | 52.04% | 93.74% | 0.78 |
| 8 | 45% | 10% | 120 | 120 | 60 | 3.42 | 1.53 | 0.57 | 33.91 | 52.98 | 59.81 | 56.17% | 94.32% | 0.89 |
| 9 | 45% | 20% | 120 | 120 | 60 | 3.78 | 1.24 | 0.39 | 18.74 | 29.27 | 24.32 | 29.85% | 98.07% | 1.20 |
| 10 | 46% | 4.5% | 120 | 60 | 60 | 1.76 | 0.54 | 0.20 | 38.63 | 60.36 | 46.44 | 63.17% | 95.54% | 1.30 |
| 11 | 40% | 10.0% | 120 | 180 | 60 | 3.17 | 1.19 | 0.89 | 31.43 | 49.11 | 46.40 | 53.94% | 91.06% | 1.06 |
| 12 | 40% | 10.0% | 120 | 120 | 60 | 3.44 | 1.23 | 0.54 | 34.15 | 53.36 | 48.01 | 56.42% | 94.57% | 1.11 |

### Examples 13-16

375 g of a 64 wt % H₂SO₄ solution was charged to a Zricodyne 702 Parr reactor along with 81 g of deionized water. The reactor was sealed and the agitation rate set to approximately 100 RPM before pressure testing and pre-heating the system to the desired reaction temperature. When the reactor reached the desired temperature, a 50 wt % solution of sucrose in water was fed into the reactor over a time period shown below in Table 2 to the specified sugar concentration. After all the sugar had been fed, the reactor was held at the reaction temperature for an additional period of time before cooling the reactor in an ice bath to room temperature for analysis. The sample was then filtered through a 1.1 μm glass mat filter to remove any solid particulate. The solids were dried at 125° C. to constant mass to obtain an accurate weight. Upon filtration, it was observed that the solid particulates did not did not adhere to the reactor internals, including the agitator and reactor side walls. The filtrate was analyzed by HPLC on a Bio-Rad Aminex column using a mobile phase consisting of 20 mM phosphoric acid augmented with 3% acetonitrile. The initial and final reactor composition of examples 13-16 and the resulting reaction mixture compositions are summarized in Table 2:

TABLE 2

| Ex- | | | | | | | | | | | LA | LA | | | |
|---|---|---|---|---|---|---|---|---|---|---|---|---|---|---|---|
| am- | % | % | M | Temp | Feed | | % LA | % FA | % Fru | % Glu | wt % | mol % | FA mol | LA Molar | LA/ |
| ple | H2SO4 | Sucrose | Sucrose | (° C.) | Time | Cook Time | (total) | (total) | (total) | (total) | Yield | Yield | % yield | Selectivity | FA |
| 13 | 40% | 10% | 0.4 | 130 | 180 | 60 | 3.79% | 0.91% | 0.08% | 0.13% | 35.32% | 55.19% | 33.48% | 56.3% | 1.65 |
| 14 | 40% | 12% | 0.5 | 105 | 120 | 120 | 4.33% | 1.78% | 0.02% | 0.15% | 35.38% | 55.29% | 53.67% | 52.5% | 1.03 |
| | | | | | | (@ 120° C.) | | | | | | | | | |
| 15 | 41% | 9% | 0.4 | 130 | 180 | 60 | 3.30% | 1.08% | 0.10% | 0.13% | 38.40% | 56.55% | 44.31% | 54.9% | 1.28 |
| 16 | 32% | 11% | 0.5 | 105 | 120 | 60 | 3.66% | 1.56% | 0.00% | 1.99% | 32.94% | 48.52% | 50.03% | 55.6% | 0.97 |
| | | | | | | (@ 120 C.) | | | | | | | | | |

### Example 17

### Distillation of Formic Acid from a Mixture of Formic Acid, Levulinic Acid, Sulfuric Acid, Water, and Unknown Impurities

To a 3 neck round bottom flask equipped with a magnetic stir bar was charged 255.60 g of a solution containing 11.12 g levulinic acid, 5.44 g formic acid, 99.43 g sulfuric acid, 139.61 g H₂O, and trace amounts of several unknown impurities The flask was equipped with a thermocouple and a short path distillation apparatus with a condenser chilled to 1 C with recirculating coolant. The distillation system was evacuated down to 40 Torr and before the kettle was heated to 45° C. The distillate was exhibited a head temperature between 31-33° C. Distillate was allowed to come overhead until the head temperature dropped below 28° C., at which point the distillation kettle was cooled to 25° C., the pressure increased to atmospheric pressure, and samples were taken from the kettle as well as distillation recovery flask. After sampling, the kettle was re-evacuated to 40 Torr and heated this time to 55° C. The procedure of distilling till the head temp falls, sampling, and redistilling at an elevated temperature was repeated until no more formic acid could be observed in the distillation kettle.

TABLE 3
Analysis of distillate and kettle samples taken during the distillation described in Example 17.

| | Kettle | | | | | | | | | % FA of | |
|---|---|---|---|---|---|---|---|---|---|---|---|
| Cut | Temp | Sample | Mass (g) | % LA | % FA | % H₂SO₄ | g LA | g FA | g H₂SO₄ | Charge | % LA of Charge |
| 1 | 65° C. | Distillate | 87.42 | — | 4.46 | — | — | 3.90 | — | 77.87 | 0.00 |
| | | Kettle | 168.18 | 6.00 | 0.48 | 55.0 | 10.09 | 0.81 | 92.50 | 16.16 | 89.73 |
| 2 | 75° C. | Distillate | 96.40 | 0.10 | 4.58 | — | 0.10 | 4.41 | — | 88.16 | 0.87 |
| | | Kettle | 159.20 | 6.49 | 0.28 | 59.3 | 10.33 | 0.45 | 91.73 | 8.97 | 91.86 |
| 3 | 80° C. | Distillate | 100.46 | 0.10 | 4.55 | — | 0.103 | 4.57 | — | 91.19 | 0.91 |
| | | Kettle | 155.14 | 6.68 | 0.23 | 59.7 | 10.36 | 0.36 | 89.83 | 7.19 | 92.16 |
| 4 | 85° C. | Distillate | 102.72 | 0.10 | 4.50 | — | 0.10 | 4.62 | — | 92.21 | 0.92 |
| | | Kettle | 152.88 | 6.59 | 0.25 | 60.6 | 10.07 | 0.39 | 89.78 | 7.69 | 89.60 |
| 5 | 90° C. | Distillate | 108.84 | 0.11 | 4.45 | — | 0.12 | 4.85 | — | 96.82 | 1.04 |
| | | Kettle | 146.76 | 6.96 | 0.00 | 63.5 | 9.87 | 0.00 | 90.04 | 0.00 | 87.77 |

### Example 18

### Vacuum Distillation of Formic Acid from a Mixture of Formic Acid, Levulinic Acid, Sulfuric Acid, Water, and Unknown Impurities with Continuous Addition of H₂O

To a 500 mL 4 neck round bottom flask equipped with a magnetic stir bar was charged 249.27 g of a solution containing 10.87 g levulinic acid, 5.31 g formic acid, 97.13 g sulfuric acid, 136.38 g H₂O, and trace amounts of several unknown impurities. The flask was equipped with a thermocouple, an addition funnel charged with 124.28 g DI H₂O, and a short path distillation apparatus with a condenser cooled to 1 C with recirculating coolant. The pressure of the system was reduced to 40 Torr before a heating mantle set to 45° C. was activated. When the solution in the flask reached approximately 42° C., distillate was observed. The head temperature fluctuated around 31-32° C. during distillation. When the distillate began to drip into the collection flask, H₂O from the addition funnel was added dropwise at roughly the same rate as the distillate was being removed. When all the H₂O from the addition funnel had been added, the pressure of the system was raised to atmospheric pressure and the system was cooled. Samples of the reaction flask mixture and distillate were taken, and the addition funnel was charged with more H₂O. The process of distilling with dropwise addition of H₂O was continued until formic acid was no longer detected in the distillation flask.

TABLE 4
Analyses of distillate and kettle samples throughout distillation described in Example 18.

| | Mass H₂O | | Mass | | | | | | | Cumulative g | % FA of | % LA of |
|---|---|---|---|---|---|---|---|---|---|---|---|---|
| Cut | Added (g) | Sample | (g) | % LA | % FA | % H2SO4 | g LA | g FA | g H2SO4 | LA in Retains | Charge | Charge |
| 1 | 124.28 | Distillate | 93.88 | 0.06 | 2.53 | — | 0.06 | 2.38 | — | 0.00 | 44.72% | 0.51% |
| | | Kettle | 276.48 | 3.80 | 0.95 | 54.10 | 10.51 | 2.63 | 149.58 | 0.00 | 49.43% | 96.69% |
| 2 | 48.62 | Distillate | 45.76 | 0.03 | 1.15 | — | 0.01 | 0.53 | — | — | 54.62% | 0.11% |
| | | Kettle | 269.46 | 3.72 | 0.67 | 49.10 | 10.03 | 1.81 | 132.30 | 0.37 | 34.08% | 95.74% |
| 3 | 51.14 | Distillate | 46.63 | 0.02 | 0.79 | — | 0.01 | 0.37 | — | — | 61.54% | 0.06% |
| | | Kettle | 263.48 | 3.66 | 0.52 | 31.30 | 9.63 | 1.38 | 82.47 | 0.73 | 25.93% | 95.34% |
| 4 | 47.25 | Distillate | 40.43 | 0.01 | 0.55 | — | 0.00 | 0.22 | — | — | 65.71% | 0.04% |
| | | Kettle | 279.62 | 3.74 | 0.44 | 42.90 | 10.47 | 1.22 | 119.96 | 1.01 | 22.89% | 105.57% |
| 5 | 47.86 | Distillate | 50.02 | 0.01 | 0.46 | — | 0.00 | 0.23 | — | — | 70.00% | 0.04% |
| | | Kettle | 244.88 | 3.72 | 0.34 | 44.00 | 9.10 | 0.84 | 107.75 | 1.24 | 15.85% | 95.08% |
| 6 | 48.63 | Distillate | 32.22 | 0.01 | 0.40 | — | 0.00 | 0.13 | — | — | 72.41% | 0.02% |
| | | Kettle | 246.85 | 3.47 | 0.25 | 45.90 | 8.56 | 0.61 | 112.40 | 1.57 | 11.43% | 93.19% |
| 7 | 101.4 | Distillate | 103.28 | 0.00 | 0.27 | — | 0.00 | 0.28 | — | — | 77.66% | 0.04% |
| | | Kettle | 236.03 | 3.55 | 0.15 | 40.70 | 8.38 | 0.35 | 99.67 | 1.80 | 6.57% | 93.70% |
| 8 | 96.82 | Distillate | 88.51 | 0.01 | 0.16 | — | 0.01 | 0.14 | — | — | 80.24% | 0.05% |
| | | Kettle | 239.65 | 3.38 | 0.09 | 43.10 | 8.10 | 0.22 | 105.54 | 1.98 | 4.06% | 92.68% |
| 9 | 97.74 | Distillate | 98.88 | 0.00 | 0.09 | — | 0.00 | 0.09 | — | — | 81.92% | 0.01% |
| | | Kettle | 229.68 | 3.51 | 0.00 | 44.80 | 8.05 | 0.00 | 109.71 | 2.18 | 0.00% | 94.10% |

### Example 19

### Formic Acid Separation from MIAK by Distillation

To a 1 L round bottom flask equipped with variac-controlled electric heating mantle, thermocouple, magnetic stir bar, pressure sensor, 1-inch×18-inch vacuum jacketed glass column packed with wire gauze packing, and magnetic bucket-type reflux control head was added 76.0 g of formic acid and 76.0 g MIAK. The still was controlled at 200 torr for a duration of 100 minutes and a reflux ratio of 6:1 reflux:collect. Bottom flask temperature ranged from 77.1° C. to 101.5° C. while the overhead temperature ranged from 60.1° C. to 61.1° C. Three fractions were collected: Fraction 1, 13.8 g, 89.187% formic acid by HPLC, Fraction 2, 18.2 g, 88.842% formic acid by HPLC, Fraction 3, 26.4 g, 88.944% formic acid by HPLC, Residual bottoms, 76.7 g, 3.261% formic acid by HPLC.

### Example 20

### Formic Acid Separation from MIBK by Distillation

To a 1 L round bottom flask equipped with variac-controlled electric heating mantle, thermocouple, magnetic stir bar, pressure sensor, 1-inch×18-inch vacuum jacketed glass column packed with wire gauze packing, and magnetic bucket-type reflux control head was added 63.47 g of formic acid and 641.55 g MIBK. The still was operated at 763 torr for a duration of 260 minutes and a reflux ratio of 6:1 reflux:collect. Bottom flask temperature ranged from 115.3° C. to 116.5° C. while the overhead temperature ranged from 97.1° C. to 114.7° C. Several fractions were collected:

| Fraction | Mass (g) | % FA by HPLC |
|---|---|---|
| 1 | 14.72 | 13.925 |
| 2 | 33.38 | 12.949 |
| 3 | 38.06 | 12.267 |
| 4 | 74.97 | 11.097 |
| 5 | 44.87 | 10.152 |
| 6 | 103.8 | 8.889 |
| 7 | 68.06 | 7.64 |
| 8 | 15.47 | 6.755 |
| Bottoms | 300.77 | 5.267 |

### Example 21

### Hydrolysis with HFCS-42

To a 1 liter Zircodyne 702 Parr kettle was charged 157.00 g H₂O and 299.99 g of an aqueous 64% H₂SO₄ solution. The kettle was placed on a Zircodyne 702 reactor equipped with a thermowell housing a J-type thermocouple, 6 blade 45° agitator, and dip tube. The kettle-reactor system was sealed with a split ring closure system before the agitator was set to approximately 123 rpm. The sealed system was pressure tested at 92 psi with N₂ to check for leaks over 10 minutes before the system was vented and the headspace re-purged with N₂. The reactor was finally blanketed with 20 psi N₂ before the kettle contents were heated to 120° C. When the reactor reached 120° C., HFCS-42 (ADM) heated to approximately 50° C. was introduced into the system through the dip tube. The HFSC-42 was added over 248 minutes until 21 wt % of solid sugar (relative to the total reaction mass) had been added. At this point, the reactor was cooled using an ice bath till the reactor contents reached approximately 30° C., the contents were filtered, and an aliquot was analyzed in a method similar to Example 1.

| | | | % | | LA Molar | FA Molar | Glucose | Mass |
|---|---|---|---|---|---|---|---|---|
| % LA | % FA | % Solids | Glucose | LA/Char | Selectivity | Selectivity | Conversion | Balance |
| 6.31 | 2.85 | 3.35% | 3.36 | 1.82 | 57.4% | 65.4% | 70.9% | 98.8% |

### Example 22

### Hydrolysis Using Recycled Raffinate

To a 1 liter Zircodyne 702 Parr kettle was charged 374.92 g of raffinate material comprising 59.04% H₂O, 36.73% H₂SO₄, 2.44% FA, 1.16% glucose, and 0.63% LA. To this raffinate was charged 85.00 g 64% H₂SO₄ and 12.49 g crystalline glucose. The kettle was placed on a Zircodyne 702 reactor system equipped with a thermowell housing a J-type thermocouple, 6 blade 45° agitator, and a dip tube. The kettle-reactor system was sealed before the agitator was set to 123 rpm. The sealed system was pressure tested at 85 psi with N₂ to check for leaks over 15 minutes before the system was vented and re-purged with N₂. The reactor was finally blanketed with 20 psi N₂ before heating the system to 126° C. The system was held at 126° C. for 60 minutes. After 60 minutes, the reactor was cooled to 30° C. and unsealed to obtain a reactor sample. With the sample taken, the system was re-sealed, pressure tested, and blanketed with N₂ as described above. After blanketing, the reactor was heated to 120° C., at which point HFCS-42 heated to approximately 50° C. was added through the dip tube over 234 minutes until a final solid sugar concentration of 19% (relative to the total reactor mass) was achieved. At this point, the reactor was cooled with an ice bath till the reactor contents reached approximately 30° C., the contents were filtered and, an aliquot was analyzed according to the methods described in Example 1.

| | | | % | % | LA Molar | FA Molar | Glucose | Mass |
|---|---|---|---|---|---|---|---|---|
| Step | % LA | % FA | Solids | Glucose | Selectivity | Selectivity | Conversion | Balance |
| Cook | 2.01 | 2.42 | 0.69 | 0.23 | 68.2 | 50.4 | 93.6 | |
| Sugar | 7.30 | 4.43 | 3.62 | 3.19 | 51.3 | 65.5 | 74.2 | |
| Feed | | | | | | | | |
| Final | | | | | 53.0 | 63.0 | | 97.2 |

### Example 23

### Extraction of Reaction Mixture with Xylenols

To a tared 15 mL centrifuge tube was charged 7.22 g of the hydrolysate produced in Example 22 and 7.26 g of a 2,4- and 2,5-xylenol solution (98% purity). The centrifuge tube was then sealed and vigorously shaken by hand for approximately 30 seconds before being centrifuged for 5 minutes at approximately 3200 rpm. The layers were then separated using a glass pipet before analyzing each layer according to the process described in Example 1.

| | | | | | | | LA | FA | Glu |
|---|---|---|---|---|---|---|---|---|---|
| | | | | | | | Component | Component | Component |
| | % LA | % FA | % Glu | LA K_{d} | FA K_{d} | Glu K_{d} | Balance | Balance | Balance |
| Aq | 1.29 | 3.46 | 0.24 | 4.92 | 0.35 | 0.02 | 96.6% | 97.0% | 100.8%* |
| Phase | | | | | | | | | |
| Org | 5.39 | 1.03 | 0.05 | | | | | | |
| Phase | | | | | | | | | |

*Greater than 100% component balance likely due to instrument error. Instrument RSD measured to 1.3%

### Example 24

### Esterification of Crude Levulinic Acid

To a 1 liter Hastelloy C-276 Parr kettle was charged 221.10 g of crude LA containing approximately 79.1% LA and 20.9% oligomers derived from the hydrolysis of HFCS-42, 138.73 g ethanol (200 proof), and 2.51 g of a 64% aqueous H₂SO₄. The kettle was then sealed onto an Hastelloy C-276 reactor equipped with a PTFE-coated thermowell housing a J-type thermocouple and a PTFE-coated 6 blade 45° agitator. The reactor-kettle system was sealed before the agitator was set to 129 rpm. The sealed reactor was then pressure tested with N₂ at 75 psi for 10 minutes before venting the headspace and re-purging with N₂. After purging, the reactor was blanketed with 20 psi N₂ before heating the system to 120° C. Once at the reaction temperature, the reactor was held at 120° C. for 150 minutes before the reactor was cooled to 30° C. using an ice bath. Once at 30° C., the reactor contents were transferred to a 3-neck round bottom flask equipped with a magnetic stir bar, at which point 4.08 g of a 33.0% aqueous NaOH solution. The neutralized reaction mixture was then distilled in three fractions using a Goodloe packed column equipped with a reflux splitter. The first fraction was distilled at atmospheric pressure using complete take-off with a pot temperature at 90-110° C. to remove residual ethanol and water. After removing as much ethanol/water mixture as possible, the reflux ratio was set at 1:1 and the pressure of the system was stepped down first to 300 Torr, then incrementally down to a final pressure of 10 Torr and the head temperature steadies between 83-85° C. The material obtained during this step-down and equilibration phase was collected as a slop cut. With a steady head temperature, the pot temperature was increased slowly to maintain distillation up to a final pot temperature of 130-135° C. The overhead material collected during this final fraction was collected as ethyl-levulinate (Et-Lev). All three fractions collected during the distillation were analyzed by GC-FID using calibration standards to quantify each component in the mixture.

| LA Molar Selectivity | LA Conversion | Et-Lev Purity* | Et-Lev Purity^{†} |
|---|---|---|---|
| 99.9% | 79.2% | 97.4%* | 99.9% |

*Determined by GC-FID, with a 1% hexanoic acid internal standard
^{†}Determined by chromatograph area %

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

1. A method to prepare levulinic acid comprising the steps:
heating a first mixture comprising water and sulfuric acid to about 80 to about 160° C. to form a solution;
adding a second mixture of water and a sugar selected from glucose and sucrose to the heated solution over a first period of time to form a liquid reaction mixture in a reactor, wherein the liquid reaction mixture comprises between 20% and 60% sulfuric acid; and
recovering levulinic acid.

2. The method of claim 1, wherein the first period of time is between 10 and 300 minutes.

3. The method of claim 1, wherein the first period of time is between 20 and 240 minutes.

4. The method of claim 1, wherein the first period of time is between 30 and 180 minutes.

5. The method of claim 1, wherein the first period of time is between 60 and 180 minutes.

6. The method of claim 1, wherein the first period of time is between 60 and 120 minutes.

7. The method of claim 1, further comprising the step of heating the reaction mixture for a reaction period at a first reaction temperature after all of the second mixture has been added to the reactor.

8. The method of claim 7, wherein the reaction period is between 10 and 300 minutes.

9. The method of claim 7, wherein the reaction period is between 20 and 240 minutes.

10. The method of claim 7, wherein the reaction period is between 30 and 180 minutes.

11. The method of claim 7, wherein the reaction period is between 60 and 180 minutes.

12. The method of claim 7, wherein the reaction period is between 60 and 120 minutes.

13. The method of claim 7, wherein the first reaction temperature is between approximately 100 to about 180° C.

14. The method of claim 13, wherein the first reaction temperature is between approximately 100 to about 160° C.

15. The method of claim 13, wherein the first reaction temperature is between approximately 100 to about 140° C.

16. The method of claim 13, wherein the first reaction temperature is between approximately 120 to about 140° C.

17. The method of claim 16, further comprising the steps:
subjecting the reaction mixture to an extraction solvent to extract levulinic acid into an extract phase;
removing the extract phase from the reaction mixture; and
recovering the levulinic acid from the extract phase.

18. The method of claim 17, wherein the extraction solvent is a phenol.

19. The method of claim 18, wherein the phenol is a halogenated phenol.

20. The method of claim 18, wherein the phenol is an alkyl phenol.

21. The method of claim 20, wherein the alkyl phenol is xylenol.

22. The method of claim 20, wherein the alkyl phenol is a mixture of xylenol isomers.

23. The method of claim 17, wherein recovering levulinic acid from the extract phase comprises distillation or crystallization.

24. The method of claim 17, wherein recovering levulinic acid from the extract phase comprises esterification with an alkanol to create a levulinic ester, followed by distillation of the levulinic ester.

25. The method of claim 17, further comprising recovering formic acid from the extract phase.

26. The method of claim 25, wherein recovering formic acid from the extract phase comprises distillation or crystallization.

27. The method of claim 25, wherein recovering formic acid from the extract phase comprises esterification with an alkanol to create a formic ester, followed by distillation of the formic ester.

28. The method of claim 1, further comprising the steps:
filtering solids from the reaction mixture, optionally after cooling;
adding a water immiscible liquid to the reaction mixture so that the reaction mixture forms first and second layers, wherein greater than 90% of the sulfuric acid is in the first layer and greater than 90% of the water immiscible liquid is in the second layer;
recovering levulinic acid and optionally formic acid from the second layer; and
recycling the first layer back to the reactor.

29. The method of claim 28, wherein the water immiscible liquid is a phenol.

30. The method of claim 29, wherein the phenol is a halogenated phenol.

31. The method of claim 29, wherein the phenol is an alkyl phenol.

32. The method of claim 31, wherein the alkyl phenol is xylenol.

33. The method of claim 28, wherein the alkyl phenol is a mixture of xylenol isomers.

34. The method of claim 28, wherein recovering levulinic acid or formic acid from the second layer comprises distillation or crystallization.

35. The method of claim 28, wherein recovering levulinic acid from the second layer comprises esterification with an alkanol to create a levulinic ester, followed by distillation of the levulinic ester.

36. The method of claim 28, wherein recovering formic acid from the second layer comprises esterification with an alkanol to create a formic ester, followed by distillation of the formic ester.

37. The method of claim 1, wherein the levulinic acid is recovered in a yield greater than 40% mol.

38. The method of claim 1, wherein the levulinic acid is recovered in a yield greater than 45% mol.

39. The method of claim 1, wherein the levulinic acid is recovered in a yield greater than 50% mol.

40. The method of claim 1, wherein the second mixture is water and glucose.

41. The method of claim 40, wherein the greater than 75% of the glucose is converted.

42. The method of claim 40, wherein the greater than 80% of the glucose is converted.

43. The method of any of claim 40, wherein the greater than 85% of the glucose is converted.

44. The method of claim 1, wherein the second mixture is water and sucrose.

45. The method of claim 1, wherein the reactor is a continuous addition batch reactor.

46. The method of claim 1, wherein the reactor is a CSTR reactor.

47. The method of claim 44, wherein the reactor is a multi stage reactor comprising at least a first reactor and a second reactor.

48. A method to prepare levulinic acid comprising the steps:
heating a first mixture comprising water and sulfuric acid to about 80 to about 160° C. to form a solution;
adding a second mixture of sugar and water to the heated solution over a first period of time to form a liquid reaction mixture in a first reactor, wherein the liquid reaction mixture comprises between 20% and 60% sulfuric acid;
heating the reaction mixture for a first reaction period at a first reaction temperature after all of the second mixture has been added to the first reactor;
feeding the liquid reaction mixture to a second reactor;
heating the reaction mixture for a second reaction period at a second reaction temperature after all of the second mixture has been added to the second reactor; and
recovering levulinic acid.

49. The method of claim 48, further comprising the step of recirculating the reaction mixture from the second reactor to the first reactor.

50. The method of claim 48, further comprising the step of adding a third mixture comprising a solution of sugar and water to the second reactor.

51. The method of claim 50, wherein the addition of the third mixture to the second reactor is simultaneous with the addition of the second mixture to the first reactor.

52. The method of claim 48, wherein the sugar is selected from the group consisting of fructose, glucose, sucrose and mixtures thereof.

53. The method of claim 52, wherein the sugar is sucrose.

54. The method of claim 48, wherein the first period of time is between 10 and 300 minutes.

55. The method of claim 48, wherein the first period of time is between 20 and 240 minutes.

56. The method of claim 48, wherein the first period of time is between 30 and 180 minutes.

57. The method of claim 48, wherein the first period of time is between 60 and 180 minutes.

58. The method of claim 48, wherein the first period of time is between 60 and 120 minutes.

59. The method of claim 48, wherein the first reaction period is between 10 and 300 minutes.

60. The method of claim 48, wherein the first reaction period is between 20 and 240 minutes.

61. The method of claim 48, wherein the first reaction period is between 30 and 180 minutes.

62. The method of claim 48, wherein the first reaction period is between 60 and 180 minutes.

63. The method of claim 48, wherein the first reaction period is between 60 and 120 minutes.

64. The method of claim 48, wherein the first reaction temperature is between approximately 100 to about 180° C.

65. The method of claim 48, wherein the first reaction temperature is between approximately 90 to about 130° C.

66. The method of claim 48, wherein the first reaction temperature is between approximately 100 to about 130° C.

67. The method of claim 48, wherein the first reaction temperature is between approximately 110 to about 120° C.

68. The method of claim 48, wherein the second reaction period is between 10 and 300 minutes.

69. The method of claim 48, wherein the second reaction period is between 20 and 240 minutes.

70. The method of claim 48, wherein the second reaction period is between 30 and 180 minutes.

71. The method of claim 48, wherein the second reaction period is between 60 and 180 minutes.

72. The method of claim 48, wherein the first reaction period is between 60 and 120 minutes.

73. The method of claim 48, wherein the second reaction temperature is between approximately 120 to about 180° C.

74. The method of any of claim 48, wherein the second reaction temperature is between approximately 120 to about 160° C.

75. The method of claim 48, wherein the second reaction temperature is between approximately 130 to about 150° C.

76. The method of claim 48, wherein the second reaction temperature is between approximately 130 to about 140° C.

77. The method of claim 48, further comprising the steps:
subjecting the reaction mixture to an extract solvent to extract levulinic acid into an extract phase;
removing the extract phase from the reaction mixture; and
recovering the levulinic acid from the extract phase.

78. The method of claim 77, wherein the extract solvent is a phenol.

79. The method of claim 78, wherein the phenol is a halogenated phenol.

80. The method of claim 78, wherein the phenol is an alkyl phenol.

81. The method of claim 80, wherein the alkyl phenol is xylenol.

82. The method of claim 80, wherein the alkyl phenol is a mixture of xylenol isomers.

83. The method of claim 77, wherein recovering levulinic acid from the extract phase comprises distillation or crystallization.

84. The method of claim 77, wherein recovering levulinic acid from the extract phase comprises esterification with an alkanol to create a levulinic ester, followed by distillation of the levulinic ester.

85. The method of claim 77, further comprising recovering formic acid from the extract phase.

86. The method of claim 85, wherein recovering formic acid from the extract phase comprises distillation or crystallization.

87. The method of claim 85, wherein recovering formic acid from the extract phase comprises esterification with an alkanol to create a formic ester, followed by distillation of the formic ester.

88. The method of any of claims 48 through 87, further comprising the steps:
filtering solids from the reaction mixture, optionally after cooling;
adding a water immiscible liquid to the reaction mixture so that the reaction mixture forms first and second layers, wherein greater than 90% of the sulfuric acid is in the first layer and greater than 90% of the water immiscible liquid is in the second layer;
recovering levulinic acid and optionally formic acid from the second layer; and
recycling the first layer back to the first or second reactor.

89. The method of claim 88, wherein the water immiscible liquid is a phenol.

90. The method of claim 89, wherein the phenol is a halogenated phenol.

91. The method of claim 89, wherein the phenol is an alkyl phenol.

92. The method of claim 91, wherein the alkyl phenol is xylenol.

93. The method of claim 91, wherein the alkyl phenol is a mixture of xylenol isomers.

94. The method of claim 88, wherein recovering levulinic acid or formic acid from the second layer comprises distillation or crystallization.

95. The method of claim 88, wherein recovering levulinic acid from the second layer comprises esterification with an alkanol to create a levulinic ester, followed by distillation of the levulinic ester.

96. The method of claim 88, wherein recovering formic acid from the second layer comprises esterification with an alkanol to create a formic ester, followed by distillation of the formic ester.
